(19) United States
(12) Patent Application Publication
(10) Pub. No.: US 2008/0167633 A1
(43) Pub. Date: Jul. 10, 2008

(54) Band-Aid With a Disinfectant Container

(76) Inventor: Roberto Vannucci, Lido di Camaiore (IT)

Correspondence Address: DAVID A. GUERRA;INTERNATIONAL PATENT GROUP, LLC, 2025 17TH AVENUE N.W., CALGARY, AB, T2M 0S7, omitted

(21) Appl. No.: 11/910,296

(22) PCT Filed: Apr. 11, 2005
(86) PCT No.: PCT/IT2005/000200

§ 371 Date: Oct. 1, 2007

(30) Foreign Application Priority Data
Apr. 11, 2005 (IT) PCT/IT2005/000200

(51) Int. Cl. A61F 13/00; A61F 13/02

(52) U.S. Cl. 604/306; 604/307

(57) ABSTRACT

The invention consists in a medication made up of an adhesive strip on one side on which a pad is applied and to which a container is integral with it (5) where a disinfectant substance or any other type is inserted in its internal part, in such a way that by opening the container in a certain point, the substance goes to be applied onto the pad itself, soaking it in case that we are dealing with a liquid substance. The part of the container (5) destined to be opened is directly in contact with the above-mentioned pad. The inferior part of the invention, containing the pad and the adhesive part, is covered by one or more strips of the plastic film or membrane, which can incorporate the above-mentioned container or tank which is removed before applying the medication to the body.

### TECHNICAL FIELD

The present invention pertains to the technical field relating to the production of sanitary products for local medication and in particular the production of band-aids or bandages made of an adhesive part on which a cotton bandage or other similar material is placed on a wound, and then fastened on the surrounding sides by an adhesive part mentioned above. In particular the invention joins together two recurrent dynamics, closely tied to each other such as the disinfecting of a small wound or a hole made when taking a blood test or when administering a medication using a syringe or in intravenous feeding or in blood transfusions followed by the corresponding protection effected by the use of common band-aids or bandages. The invention therefore consists in a medication or dressing and is intended for any product used for therapeutic aims.

### BACKGROUND ART

As of today the aim has always been the usage of two distinct operations, whether effected by specialized personnel in the sanitary structures or in the sphere of small personal domestic operations. The consolidated methodology consists in fact in disinfecting the wound with special products, generally liquid, which subsequently becomes protected by the application of a band-aid that consents in isolating the wound against germs, bacteria or whatever external substances that are potentially infective. In the specific case in which it is preferable for the wound to have a prolonged contact with the disinfectant substance a band-aid/bandage/gauze is generally interposed between a cotton swab soaked in a disinfectant. This frequently happens, for example, on wounds caused by needles or syringes, whether they are used for blood tests or for injections. Such prophylaxis acquires greater importance in the case of intravenous injections, which if not cleaned properly, can become a probable vehicle of infection.

It is of common experience to leave such operations using cotton swabs soaked in alcohol in contact with the concerned parts, perhaps held together in position by means of a band-aid or bandage placed to hold the cotton swab. Even for this simple intervention it is indispensable to always have the disinfectant liquid, cotton and bandage ready; this can appear to be taken for granted in the context of the doctor's office or in a hospital structure, however still maintaining an implicit relative complexity. Even more so, such different medical assumptions are taken to be important in the case of pathologies that implicate the necessity of frequent injections, which are not always effected in medical structures but which however oblige you to always have the necessary accessories with intrinsic evident inconvenience which certainly don't improve the quality of the patient's life.

Furthermore we must point out that there is a whole potential use in this field in which the immediate complexity in finding such protective systems often brings us to disregard the indispensability with personal and social consequences that such conduct involves.

Besides, even in the professional field the simplicity of such operations makes the doctors and nurses work more secure and productive that due to the actual technique they are obliged to pass a notable lapse of time proceeding with the interventions of disinfecting.

Therefore the traditional band-aids don't consent in applying any disinfectant or any other product on a particular part of the body. Each time a substance must be applied to the wound and sealed with a cotton swab and then a band-aid applied onto the cotton swab. This procedure also happens when a cream or any other lotion are applied on a particular part of the body not only for therapeutic but also for aesthetic reasons.

### DISCLOSURE OF INVENTION

The aim of the present invention is to avoid these and other inconveniences, furnishing a bandage or band-aid made up of a tank containing the substance to be applied on a particular part of the body, of simple and immediate use, of reduced dimensions and of contained costs.

The advantages which derive from the present invention consists in that it is possible with a single object, by means of a simple manual operation, to disinfect with safety measures and at the same time protect any type of small wounds without the necessity of finding cotton and a disinfectant besides the bandage; that it is possible to apply any kind of substance on an area of the body, pre-loading the substance in the tank integral with the bandage; that it essentially reduces the time necessary to disinfect the wound or apply the substance in that the operation comes about using a single object; that it is particularly practical when you are not at home or not in a sanitary structure, in that with a single object you can obtain the result of sealing the wound and disinfecting it too.

These and further advantages and characteristics of this present invention will be better understood by any technical person in the field by the description which follows and with the help of the attached designs given as illustrative titles.

The invention spoken of here is made up of a device having the form of a band-aid or bandage, which can have any form and dimensions, similar to the present existing band-aids. Even the protective system of the internal surface, normally made up of adhesive membranes or films destined to protect the pad which is in contact with the wound; it is of a common plan and made of the same plastic material. But this band-aid presents a thicker pad with respect to the traditional band-aids and joined to this pad a capsule or tank is provided containing the substance which is to be applied, whether it be a disinfectant, a cosmetic cream or any other type of substance.

Reduced to its essential structure and in reference to the attached figures in the designs, a medication, in conformity with the present invention, includes an adhesive strip on one side equipped with a pad characterized by the fact that it includes a small container (5) in which any type of a substance is put into it (4) integral with the pad (2), so that in opening the container (5) in a certain point the substance comes out going directly onto the pad or anyway soaking it.

The pad is in cotton, gauze, cotton swab or in any other suitable material to seal a wound or anyway made in such a way as to be able to cover and dress a part of the body. Its surface can be of variable dimensions, corresponding to that of the actual band-aids with pads (about 2 cm.×1 cm.) or of greater dimensions, according to the use that the medication is expected to cover.

In order to cover small wounds or small pieces of skin the dimensions mentioned-above can be sufficient or slightly larger dimensions such as 3 cm.×2 cm. In order to cover a wider surface such as in the case where the medication is used to apply creams on abrasions or lotions even for aesthetic purposes on wider parts of the skin, the pad (2) can have dimensions of 4 cm.×5 cm. or even larger.

The pad (2) has an advantage, independently from the dimensions of the surface, because it has a larger thickness in respect to the common band-aids traditionally used, in that the pad in object, which was invented not only serves the purpose of covering the wound or the part of the body to medicate but also releases the substance which has been absorbed in such a way that it penetrates into the skin. In the case in which the substance is a cream this will not penetrate into the pad but will remain on the surface and therefore through the application of the medication will penetrate directly into the skin.

The substance contained in the container (5) can be of any type as long as it is fluid and for fluid we mean any form of cream, jelly or any liquid substance of its kind.

The invention has been studied for the purpose of using a disinfectant substance which is contained in the internal part of the capsule or cap. This disinfectant comes out and soaks the pad which in turn becomes the disinfectant itself. Therefore the user can directly apply the medication as in the use of a traditional band-aid, removing the protective film or membrane and placing it on the wound leaving the adhesive strip to seal the medication on the skin. In such a solution the invention consents in particular to be utilized after taking blood tests to seal and disinfect the wounds, making the operation possible with a single object and with a simple gesture.

This container (5) can assume various forms and dimensions but it is integral with the medication and in particular in contact with the pad at least with the part that opens up and the substance comes out the from it (4).

The container can assume the form of a semi-circle, of a rectangle, of a circle or of any other form. The dimensions are contained and reproduced in accordance to the dimensions of the medication.

In the present solution the invention is made of a cap or capsule (5) able to contain a substance (4) integral with a duct (6) oriented in the direction of the pad (2) in which the inferior part is sealed with a protective film or membrane which is welded (7).

This above mentioned film or membrane occludes the capsule which is equipped with a pre-incision (9), effected in correspondence to the above-mentioned duct, in a transversal way which provokes a weakness in the thickness of the material of which it is made, having a specific task to make it break. In fact, using a slight inflexion exerted perpendicularly to the duct, you obtain the breaking of the duct with the consequent releasing of the product onto the soaked pad placed in the interior of the band-aid.

Advantageously at least the part of the duct (6) on which the pre-incision is made (9) is found on the pad in such a way that in breaking this part and opening the duct in a certain point, the substance contained in it goes directly to soak the pad.

Advantageously the whole inferior welded part (7) is in contact with the internal surface of the band-aid and preferably on the top of the pad (2). This above-mentioned part (7) is completely incorporated in the plastic film or membrane (3).

Advantageously the capsule or cap (5) and the above-mentioned inferior part (7) found on the external part of the pad (FIG. 7, 8, 9) or on the external part of the band-aid, being only the terminating or ending part of the duct (6) with the pre-incision (9) positioned above the pad itself. In such a solution only a part of the inferior part (7) is integral with the plastic film or membrane (3).

In a different effected solution, the capsule (5) contains a pivot, pin or a welded cusp or die-casting on the cap itself (10), positioned in the centre or lateral side of the cap and integral with it on the upper part, whose pressure permits the point to perforate the lower flat part of the container (10A) releasing the substance onto the pad (2). The capsule (5) is however integral with the plastic film or membrane (3) in a way that its inferior part (10A) is a part of the above-mentioned plastic film or membrane.

The cap or capsule (5) and the plastic film or membrane (3) can be realized as a single body, for pressing or die-casting or they can be two distinct bodies subsequently joined together between themselves.

In a further effected solution, the invention includes a cap with a micro-valve with a little piston and a membrane in its interior (11) situated in an adjacent position to the pad of the band-aid; the compression of the cap or capsule provokes the primer in the internal valve that, thanks to its conformation, restores the fluid from the tank by pressure, carrying it onto the pad destined to come into final contact for medication with the wounded part. In such an actual solution the cap is made up of a welded sack (12) containing disinfectant liquid, jelly or cream.

Advantageously the blister (12) which is welded to the protective film or membrane (3) and containing a micro-valve is circularly bound on the inferior circular sector of its little piston (13), which is activated through compression and determinates the lowering of the little piston (3) and at the same time putting pressure into the liquid (15) in the interior of the sealed chamber which determines the inevitable conveying by force in the direction of the pad (2) of the band-aid (1).

Advantageously the container (5) is incorporated in the plastic film or membrane (3) which protects the adhesive strip (1) containing the pad (2) band-aid or bandage. When the pad (2) has been soaked in the substance (4) contained in the container (5) it is sufficient to pick up the plastic film or membrane and remove the sack (5) along with the above-mentioned film or membrane.

Advantageously the medication in object is sold in a single sealed blister thus avoiding contact with germs or other pathogenic elements. Because of this the product presents itself, even from an hygienic point of view, to be much better than the actual band-aids which exist that normally assume the form of rolls or even when they are sealed singularly must be applied to the part of the body after a swab of cotton soaked in a disinfectant substance or cream which are probably exposed to the aggressive agents present in the air.

The container (5) can be integrated with the medication and be part of it in a single form or else can be separated from it.

The invention can be achieved by means of already existing machinery, that, operating together as described above, achieve an innovation thus not necessitating a particular construction of machinery nor the running in nor the focussing in the realization and/or the functioning of the invention. This, thanks to its simple construction, has a reduced cost of production, especially in the case of highly serial productions.

In practice however the details for the execution can vary in an equivalent way in its form of dimensions and disposition of the elements, the nature of the materials used without otherwise leaving the circle of the idea of solution used and therefore remaining in the limits of the protection granted for the present patent for industrial invention.

### BRIEF DESCRIPTION OF DRAWINGS

FIG. 1 shows a view of the plan and indicates the lateral section (x-x) of the band-aid in which we can see the extreme compactness of the object and joined to the following FIG. 2, showing the bursting of the same, the diverse components are indicated: the detailed one (1) represents the base of the invention having a similar conformity to a common band-aid, equipped with a central pad (2) destined to be in contact with the wound. The two strips of the inferior protection (3, 3A), have a protective function of the pad and of the adhesive part, but on the longer side or part (3) the cap is incorporated (5) containing the disinfectant or the substance to be applied (4). A special diaphragm (7), welded in the lower part of the film or membrane is provided to contain the fluid in correspondence to the container (5) and to the duct (6) through which the disinfectant is conveyed (4), prior to the primer system obtained by means of a slight practical manual bending (f) and (f1), which acts on a transversal pre-incision (9) accomplished on the welded film or membrane (7), destined therefore to break and communicate with the duct (6) with the pad situated below (2) thus soaking the pad with the liquid. Once we have obtained this aim, it will be sufficient, as with the common band-aids, to dispose of the protective film or membrane together with the tank and apply the disinfectant band-aid to the wound in the traditional way.

FIG. 3 illustrates another constructive modality of the same functional concept. The figure shows the identical unity of the product, with a transversal view and plan and with a detail of the cap indicated. The following FIG. 4. shows an exploded view of the various parts of the design. Even though such a version is composed of a common band-aid (1) with a respective pad (2) equipped with inferior protective strips (3, 3A), the cap (5) which contains the product (4) is much more compact. The releasing of the product comes through pressure (P) on the pin (10) solidly anchored to the container (5). The flange of the base of the container (7), circular and flat is welded to a protective film or membrane (3), and conveniently positioned in correspondence to the pad (2). The film or membrane (3), once perforated by the point of the pin (10), permits the releasing of the disinfectant onto the pad (2). Once having obtained this, it will be sufficient to remove the two films or membranes with the tank incorporated in it and apply the band-aid on the wound.

FIG. 5 shows in an exploded view of the various parts of the design a further variation of the activating system of the invention. In this case the particular primer system will consent to the utilization of fluid solutions, and not only limiting it to liquids as in the case of the previous applicable examples. Such a system not only uses the opening of a duct which comes into contact between the tank and the pad, ideal for liquids, but also consents to the extruded interaction of the manual pressure exerted on the tank, therefore also permitting a thicker solution like a cream, a jelly or anything else similar to cover the part destined to come into contact with the wound, that is the pad of the band-aid. The priming device is made up of a micro-valve (11) inserted on the part that rests on the pad of a thermo-welded blister (12) of the protective film or membrane (3). The following FIG. 6 illustrates in the plan and in the relative section together with the relative detail “D” the dynamics of the cylindrical little piston or a primer of any other form (13), whose activation by compression determines the opening of the micro-valve consequently lowering the little piston from its lodging, and opening in a radial and circular way in order to let out the fluid (15), that pressed into the box/tank of the blister (12), reverses onto the surface of the pad (2), thus offering an active medication to the part destined to come into contact with the wound and not, as in the actual band-aids, limiting it only to the protective side. The relative detail “D” enlarged, thus illustrates this specific system.

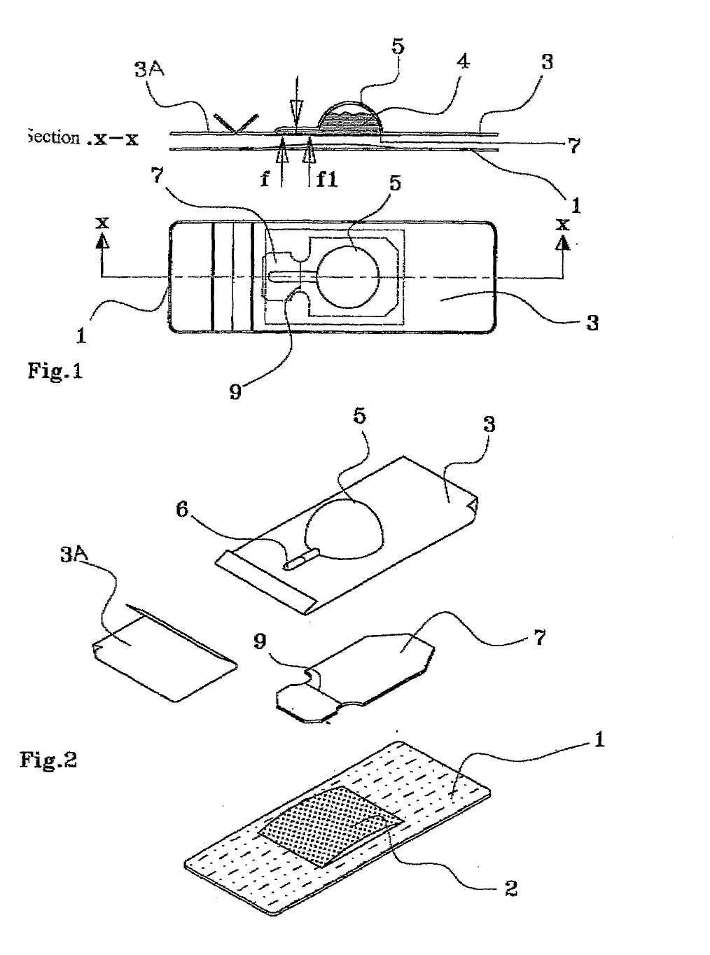

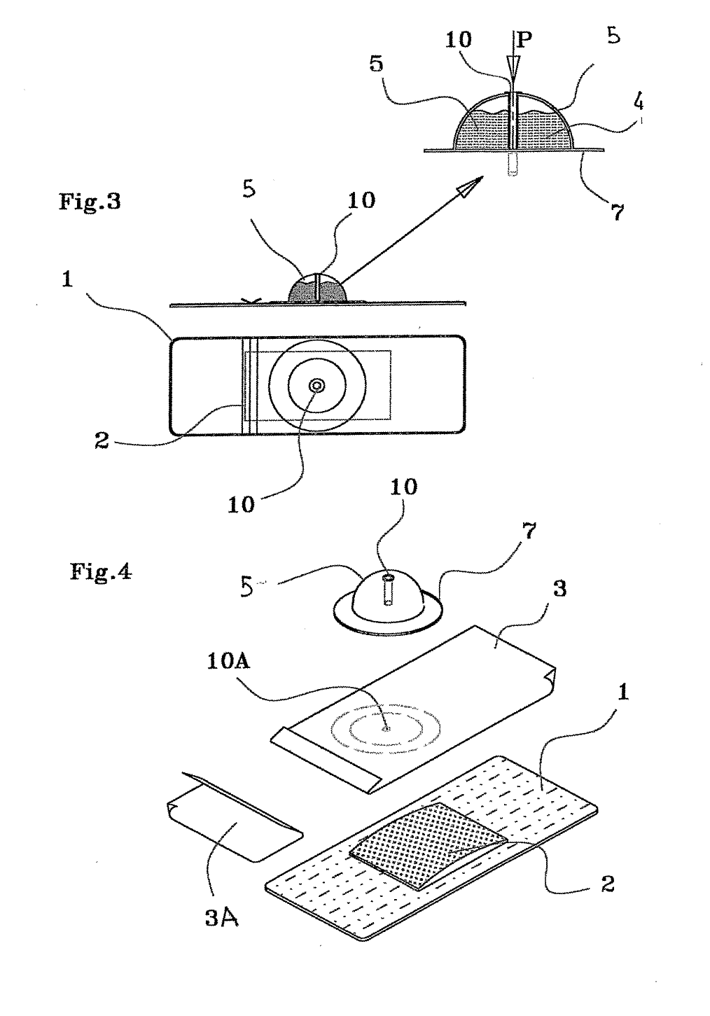

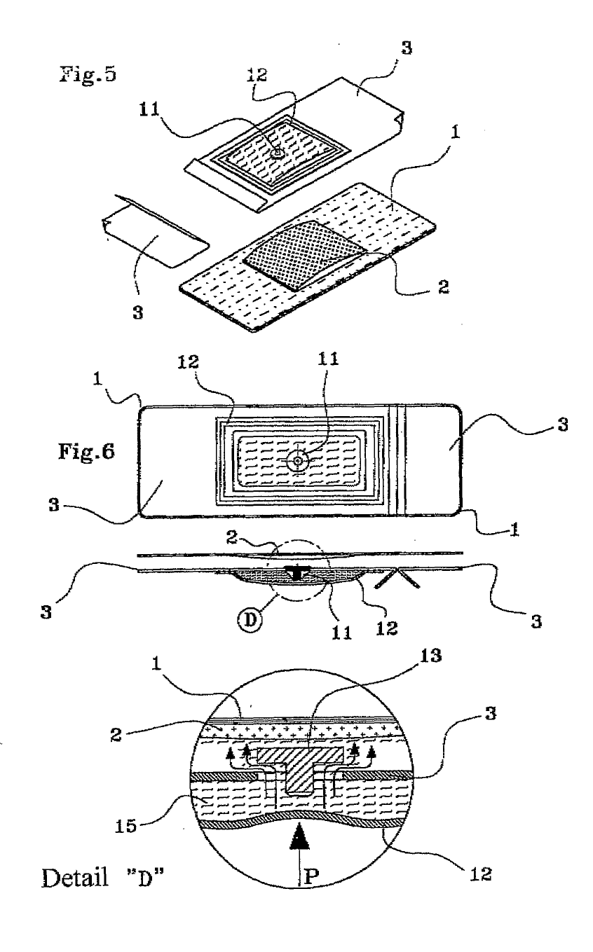

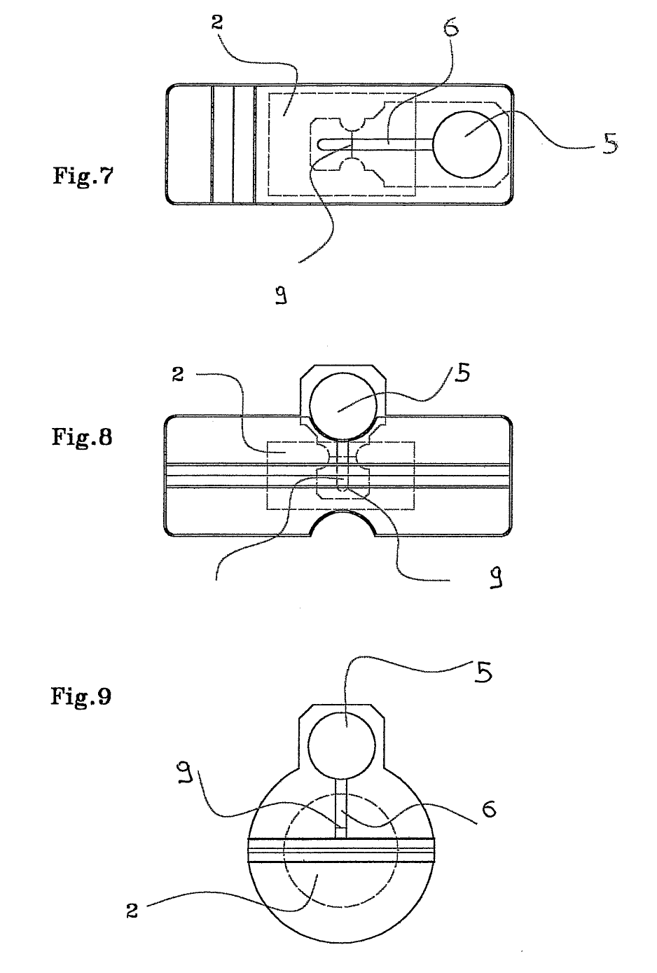

The following FIG. 7 illustrates, in the plan, some of the possible multitudes of positioning the tank containing the liquid, cream, jelly or any other medication that in conformity with the innovating idea of the invention, adopts itself well with the various existing typologies of the band-aid, as also illustrated in the following FIGS. 7 and 8 independently from their geometric form but however using the same principle of the breaking primer (9) with external tank (5), which permits passage of the medication contained in the connecting duct (6) which puts into communication the fluid with the pad (2).

1-19. (canceled)

20. A medical device for containing and applying a substance to an area of skin or a wound, said medical device comprising:
a band-aid strip having on one side an adhesive strip and a pad; and
a membrane having a container adapted to contain a substance therein, said membrane being positionable over said pad and said adhesive strip of said band-aid strip;
wherein said container being adapted to dispense said substance onto said pad of said band-aid strip.

21. The medical device according to claim 20, wherein said pad is selected from the group consisting of cotton, gauze, cotton swab, and a suitable material to seal a wound or however in such a way as to be able to cover and bandage a part of the body.

22. The medical device according to claim 20, wherein said substance contained in said container is a fluid selected from the group consisting of a cream, a jelly, and a liquid.

23. The medical device according to claim 20, wherein said substance is a disinfectant in a liquid state.

24. The medical device according to claim 20, wherein said container further comprising a duct oriented in the direction of said pad and in communication interior of said container, said duct having an inferior part sealed by a protective film having a pre-incision, said pre-incision being adapted in such a way that acting thereon produces an opening in that point of said duct and therefore dispensing said substance onto said pad.

25. The medical device according to claim 24, wherein said protective film is welded to said container and to said duct.

26. The medical device according to claim 24, wherein said pre-incision is effected in correspondence to said duct in a transversal way such as to provoke a weakness in the thickness of the material of which it is made.

27. The medical device according to claim 24, wherein at least a part of said duct on which said pre-incision is located is to be found on said pad, and wherein an inferior part of said protective film is in contact with an internal surface of said band-aid strip and above said pad.

28. The medical device according to claim 20, wherein said container further comprising a pin integral with said container and extending interiorly therefrom permitting its opposite point to puncture a lower flat part of said container releasing said substance onto said pad.

29. The medical device according to claim 28, wherein said container is integral with said membrane in such a way that said lower flat part is a part of said membrane.

30. The medical device according to claim 20, wherein said container and said membrane are two distinct bodies subsequently joined together between themselves.

31. The medical device according to claim 20, wherein said container further comprising a mechanical breaking micro-valve with a piston in its interior located in adjacent position to said pad, wherein said micro-valve, if compressed by pressure applied to the exterior of said container, causes said piston in said micro-valve to move away from its lodging and create an opening thereby allowing said substance to flow from the interior of said container onto said pad.

32. The medical device according to claim 31, wherein said container is a welded sack containing said substance therein.

33. The medical device according to claim 32, wherein said sack is welded to said membrane, and wherein said micro-valve is circularly bound on a circular sector of said piston, whose activation by means of compression determines the opening of said micro-valve as well as the contemporary placing of said substance by means of pressure inside said sack which determines the forced conveying in direction onto said pad.

34. A medical device comprising:
a band-aid strip having on one side an adhesive strip and a pad; and
a membrane having a container adapted to contain a substance therein, a duct oriented in the direction of said pad and in communication interior of said container, and a protective film having a pre-incision and adapted to seal an inferior part of said duct, said membrane being positionable over said pad and said adhesive strip of said band-aid strip, said pre-incision being adapted in such a way that acting thereon produces an opening in that point of said duct and therefore dispensing said substance onto said pad;
wherein said container being adapted to dispense said substance onto said pad of said band-aid strip.

35. The medical device according to claim 34, wherein said pre-incision is effected in correspondence to said duct in a transversal way such as to provoke a weakness in the thickness of the material of which it is made.

36. The medical device according to claim 34, wherein at least a part of said duct on which said pre-incision is located is to be found on said pad, and wherein an inferior part of said protective film is in contact with an internal surface of said band-aid strip and above said pad.

37. A medical device comprising:
a band-aid strip having on one side an adhesive strip and a pad; and
a membrane having a container adapted to contain a substance therein, and a member located in the interior of said container and moveable upon pressure acting upon said container, said membrane being positionable over said pad and said adhesive strip of said band-aid strip;
wherein said container being adapted to dispense said substance onto said pad of said band-aid strip.

38. The medical device according to claim 37, wherein said member a pin integral with said container and extending interiorly therefrom permitting its opposite point to puncture a lower flat part of said container releasing said substance onto said pad.

39. The medical device according to claim 37, wherein said member a piston located in adjacent position to said pad, wherein said piston, if compressed by pressure applied to the exterior of said container, causes said piston to move away from its lodging and create an opening thereby allowing said substance to flow from the interior of said container onto said pad.
